(19) United States
(12) Patent Application Publication
(10) Pub. No.: US 2020/0289089 A1
(43) Pub. Date: Sep. 17, 2020

(54) ULTRASOUND DEVICE INCLUDING A DETACHABLE ACOUSTIC COUPLING PAD

(71) Applicant: EchoNous, Inc., Redmond, WA (US)

(72) Inventors: David Nelson, Redmond, WA (US); Nikolaos Pagoulatos, Kirkland, WA (US)

(21) Appl. No.: 16/818,813

(22) Filed: Mar. 13, 2020

Related U.S. Application Data
(60) Provisional application No. 62/819,014, filed on Mar. 15, 2019.

(51) Int. Cl. A61B 8/00; A61B 5/0408

(52) U.S. Cl. CPC A61B 8/4411; A61B 5/04085; A61B 8/4416; A61B 2562/066; A61B 8/4281; A61B 8/4455; A61B 8/4236

(57) ABSTRACT

A device obtains ultrasound signals with ultrasound sensors without using ultrasound coupling gels on the face of the device. One such device includes an acoustic coupling pad that is placed on the ultrasound sensors. The acoustic coupling pad replaces conventional water-based ultrasound sensing gels to obviate the need for using such gels that may cause an electrical short circuit between electrode leads of electrocardiogram sensors positioned adjacent to the ultrasound sensors in the face of the device.

### BACKGROUND

### Technical Field

The present disclosure pertains to physiological sensing devices, and more particularly to such devices for acquiring ultrasound data using an acoustic coupling between the device and a patient.

### Description of the Related Art

Ultrasound imaging is typically performed in a clinical setting, by trained ultrasound experts, utilizing ultrasound systems or devices that are specifically designed to acquire ultrasound data. In order to enhance the reception of this physiological data, an ultrasound transmission gel or ultrasound gel is usually applied at the face of the ultrasound sensor device or on the skin of the patient by a physician or other clinician. The ultrasound gel is typically an electrically conductive material, such as a water-based gel, and when ultrasound gels are applied to an area of skin of the patient that covers a target tissue area, it eliminates any air between the sensor and the skin. The gel forms an acoustic pathway between the sensor and the skin and facilitates the transmission of ultrasound signals.

### BRIEF SUMMARY

The present disclosure provides a multifunctional device capable of sensing ultrasound data and electrocardiogram (ECG) data with the same device.

In various embodiments, the present disclosure provides a device that incorporates an acoustic coupling pad capable of providing an acoustic pathway between the device and a patient, which facilitates acoustic coupling without the use of conventional ultrasound sensing gels.

Moreover, in various embodiments, the present disclosure provides an acoustic coupling pad that can be attached at the sensor face of an ultrasound device at a position that is spaced apart from one or more ECG sensor leads on the sensor face. The acoustic coupling pad provides acoustic coupling between the device and a patient during a diagnostic process, while preventing the ECG sensor leads from being electrically connected or short-circuited to each other through the acoustic coupling pad.

Additionally, in various embodiments, the present disclosure provides a general use acoustic coupling pad that can be easily attached and detached at the sensor face of any medical devices without having to use sensing gels that may discomfort the patient.

In an embodiment, a device is provided that includes an ultrasound sensor on a sensor face of the device, an electrocardiogram (ECG) sensor on the sensor face of the device, and an acoustic coupling pad on the ultrasound sensor, the ECG sensor being spaced apart from the acoustic coupling pad. The ultrasound sensor includes an ultrasound transducer array and an ultrasound lens on the ultrasound transducer array. The acoustic coupling pad is removably attached to the ultrasound lens.

In another embodiment, an acoustic coupling pad for an ultrasound device is provided that includes an acoustically conductive body having a first surface and a second surface opposite the first surface, a biocompatible coating layer on the first surface, and an adhesive layer on the second surface. The biocompatible coating layer includes biocompatible silicone. The thickness of the acoustic coupling pad is less than 10 mm. The acoustically conductive body includes a synthetic rubber. The acoustic coupling pad may be attached to a backing. The acoustic coupling pad may be removably secured to the backing by the adhesive layer.

In yet another embodiment, an ultrasound probe is provided that includes a housing, a sensor face exposed at one end of the housing, an ultrasound transducer array, an ultrasound lens on the ultrasound transducer array and adjacent to the sensor face, and an acoustic coupling pad removably attached to the ultrasound lens. The ultrasound lens defines at least a portion of the sensor face of the ultrasound probe, and the acoustic coupling pad extends outwardly beyond the sensing face. The ultrasound lens is recessed with respect to the sensor face of the ultrasound probe.

### BRIEF DESCRIPTION OF THE SEVERAL VIEWS OF THE DRAWINGS

For a better understanding of the embodiments, reference will now be made by way of example only to the accompanying drawings. In the drawings, identical reference numbers identify similar elements or acts. The sizes and relative positions of elements in the drawings are not necessarily drawn to scale. For example, the shapes of various elements and angles are not necessarily drawn to scale, and some of these elements may be enlarged and positioned to improve drawing legibility. Further, the particular shapes of the elements as drawn are not necessarily intended to convey any information regarding the actual shape of the particular elements, and may have been solely selected for ease of recognition in the drawings.

FIG. 1 is a perspective view illustrating a device having an ultrasound sensor, an electrocardiogram (ECG) sensor, and an acoustic coupling pad, in accordance with one or more embodiments of the present disclosure.

FIG. 2 is an enlarged perspective view of a sensor portion of the device shown in FIG. 1 without the acoustic coupling pad, in accordance with one or more embodiments.

FIG. 3 is an enlarged perspective view of the pad portion and the sensor portion of the device shown in FIG. 1, in accordance with one or more embodiments.

FIG. 4 is a cross-sectional view taken along the cut-line 4-4 of FIG. 3, illustrating further details of the pad portion and the sensing portion of the device, in accordance with one or more embodiments.

FIG. 5 is a perspective view of an acoustic coupling pad, in accordance with one or more embodiments.

### DETAILED DESCRIPTION

In the following description, certain specific details are set forth in order to provide a thorough understanding of various disclosed embodiments. However, one skilled in the relevant art will recognize that embodiments may be practiced without one or more of these specific details, or with other methods, components, materials, etc. In other instances, well-known structures associated with ultrasound medical devices and electrocardiogram sensors have not been shown or described in detail to avoid unnecessarily obscuring descriptions of the embodiments.

Unless the context requires otherwise, throughout the specification and claims that follow, the word “comprise” and variations thereof, such as, “comprises” and “comprising” are to be construed in an open, inclusive sense that is as “including, but not limited to.” Further, the terms “first,” “second,” and similar indicators of sequence are to be construed as interchangeable unless the context clearly dictates otherwise.

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

As used in this specification and the appended claims, the singular forms “a,” “an,” and “the” include plural referents unless the content clearly dictates otherwise. It should also be noted that the term “or” is generally employed in its broadest sense that is as meaning “and/or” unless the content clearly dictates otherwise.

Further, the break lines in the drawings are used to indicate that there are more elements present but are omitted for the sake of simplicity.

Frequently used methods of diagnosis in medicine for physiological assessment, e.g., of the cardiothoracic cavity, include sonography, auscultation, and electrocardiography. These methods of diagnosis provide different kinds of information usable to assess the anatomy and physiology of the organs present in a region of interest, e.g., the cardiothoracic cavity.

Medical ultrasound imaging (sonography) has been one of the most effective methods for examining both the heart and the lungs. Ultrasound imaging provides anatomical information of the heart as well as qualitative and quantitative information on blood flow through valves and main arteries such as the aorta and pulmonary artery. One significant advantage of ultrasound imaging is that, with its high frame rate, it can provide dynamic anatomical and blood flow information, which is vital for assessing the condition of the heart, which is always in motion. Combined with providing blood flow information, ultrasound imaging provides one of the best available tools for assessing the structure and function of heart chambers, valves, and arteries/veins. Similarly, ultrasound imaging can assess fluid status in the body and is the best tool in assessing pericardial effusion (fluid around the heart).

In the case of lungs, ultrasound imaging provides information on the anatomical structure of the lungs with the ability to show specific imaging patterns associated with various lung diseases and with an ability to assess fluid status around the lung and within individual compartments of the lung including the assessment of pericardial effusion.

Auscultation allows for assessing the physiological condition and function of organs such as the heart and lungs by capturing audible sounds that are produced by or otherwise associated with these organs. The condition and function of these organs, or other organs as the case may be, can be evaluated based on clinical information indicating how different sounds are associated with various physiological phenomena and how the sounds change for each pathological condition.

Electrocardiography (ECG) is focused on the heart by capturing the electrical activity of the heart as it is related to the various phases of the cardiac cycle. The condition and function of the heart may be evaluated based on clinical knowledge indicating how the electrical activity of the heart changes based on various pathological conditions.

In order to sense the above mentioned physiological data of a patient, some medical sensing devices incorporate various sensors in one device to conveniently detect multiple data at the same time. Some devices are capable of detecting both ultrasound data and ECG data using the same device. For example, in various embodiments provided herein, an ultrasound device may include one or more ECG leads spaced apart from an ultrasound sensor on a sensor face of the device. In conventional ultrasound imaging, an ultrasound transmission gel or ultrasound gel is typically applied to the sensor or the patient to enhance reception of ultrasound signals. However, since ultrasound gels are typically electrically conductive water-based gels, such ultrasound gels could electrically connect or short circuit the ECG leads in devices having ECG leads arranged on or near the sensor face. When this occurs, the ECG data cannot be acquired correctly and the signals are likely to have noise or sometimes no signal at all.

The present disclosure provides devices and methods in which ultrasound and ECG signals may be acquired by a single handheld device that does not utilize any ultrasound sensing gels.

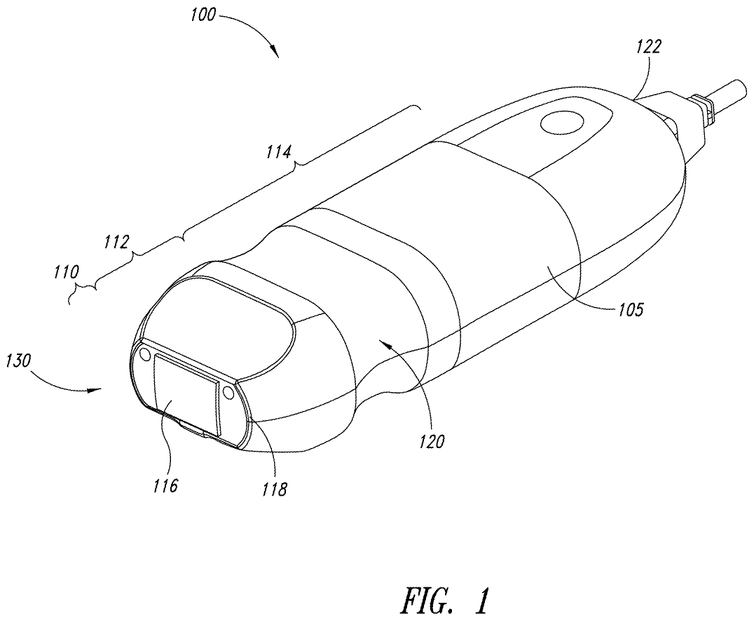

FIG. 1 is a perspective view illustrating a device 100 having an ultrasound sensor, an electrocardiogram sensor, and an acoustic coupling pad, in accordance with one or more embodiments of the present disclosure.

The device 100 can be connected to another device having a display screen to display relevant data acquired from diagnosing a patient. In some embodiments, the device 100 may include various circuitries, such as microprocessors, signal/data processing circuitry, etc., to process the acquired information (e.g., physiological data including ultrasound data or electrocardiography data of a patient). Additionally, or alternatively, the device 100 may transmit the acquired physiological data of the patient to another device for processing the data acquired by the device 100. These connected devices may include microprocessors, various signal/data processing circuitries, or the like to process the physiological data of the patient. For example, the connected electronic device may include, but is not limited to, mobile phones, handheld devices, a personal computer (PC), notebook computers, laptops, tablet PCs, and any other devices capable of data processing.

In operation, a user may place the sensor face 130 of the device 100 in a desired location on a patient's body. Once suitably positioned, the device 100 may be operated to acquire signals using one or more sensors on the sensor face 130, such as auscultation sensors (not shown), ECG sensors (not shown), and ultrasound sensors (not shown). In some embodiments, the signals acquired from one or more of the auscultation sensors, the ECG sensors, and the ultrasound sensors may be simultaneously acquired and synchronized with one another. With various sensors positioned on the sensor face 130, the device 100 may be utilized to obtain various physiological data with one scan of a target area or region of the patient.

The device 100 may include a housing 105 that forms an exterior of the device 100. The housing 105 may house any microprocessors, for example, signal processing circuitry, data processing circuitry, digital signal processors (DSP) for digital signal processing, and various sensors for sensing physiological data of the patient. In some embodiments, the housing 105 may include a pad portion 110, a sensor portion 112 and handle portion 114.

The pad portion 110 is near a first end 118 of the housing 105. The first end 118 is proximate to the sensor face 130, which will be in contact with the patient during use of the device 110. The second end 122 is at an opposite side of the housing 105 than the first end 118. The handle portion 114 is between the first end 118 and the second end 122 of the housing 105 to provide a convenient grip for the person using the device 100. The sensor portion 112 is between the pad portion 110 and the handle portion 114. The sensor portion 112 includes various sensors for acquiring physiological data from the patient. For example, the sensor portion 112 may include ECG sensors for acquiring electrocardiography data of the patient. The sensor portion 112 may also include ultrasound sensors for acquiring ultrasound data. In addition, the sensor portion 112 may include auscultation sensors for acquiring auscultation data. In FIG. 1, the handle portion 114 is shown as being positioned between the second end 122 and the sensor portion 112. However, in different embodiments, the location of the sensor portion 112 and the handle portion 114 can change according to design needs or objectives and does not necessarily have to be fixed at certain locations.

The pad portion 110 extends outwardly from the first end 118 of the housing 105 and the sensor portion 112. The pad portion 110 is generally located close to the first end 118 so that the pad portion 110 may directly contact the skin surface of the patient during use of the device 100. For example, one side of the pad portion 110 is in direct contact with the sensor portion 112 and the other side may be in direct contact with the patient. It will be explained later on in detail that this pad portion 110 having an acoustic coupling pad 116 may serve as a replacement for conventional ultrasound gels, which are typically water-based gels to transfer the acquired acoustic signals with low or no acoustic loss.

The handle portion 114 is a portion of the housing 105 that is gripped by a user to hold, control, and manipulate the device 100 during use. The handle portion 114 may include gripping features, such as one or more detents 120, and in some embodiments, the handle portion 114 may have a same general shape as portions of the housing 105 that are distal to, or proximal to, the handle portion 114. In general, the handle portion 114 refers to a portion of the housing 105 that is located between the sensor portion 112 and the second end 122 of the housing 105, which will be described in further detail later herein.

In some embodiments, the housing 105 may further surround internal electronic components and/or circuitry of the device 100, including, for example, electronics such as driving circuitry, oscillators, beamforming circuitry, filtering circuitry, and the like. The housing 105 may be formed to surround or at least partially surround externally located portions of the device 100, such as the sensor face 130, and the housing 105 may be a sealed housing, such that moisture, liquid or other fluids are prevented from entering the housing 105. The housing 105 may be formed of any suitable materials, and in some embodiments, the housing 105 is formed of a plastic material. The housing 105 may be formed of a single piece (e.g., a single material that is molded surrounding the internal components) or may be formed of two or more pieces (e.g., upper and lower halves) which are bonded or otherwise attached to one another.

The pad portion 110 may include an acoustic coupling pad 116 that is placed on a portion of the sensor face 130. The acoustic coupling pad 116 may be positioned to partially cover the sensor face 130, such that the sensors located near the sensor face 130 are spaced apart from and do not directly contact the patient (e.g., the patient's skin) during use of the device 100. For example, instead of the sensor face 130 directly contacting the patient's skin, the acoustic coupling pad 116 placed in between the patient and the sensor face 130 can acoustically couple the patient (more specifically the body part of the patient that is being imaged by the device) with the device 100 during use. The acoustic coupling pad 116 may serve as an acoustic pathway for physiological signals to be transmitted and received by the ultrasound sensor of the device 100 during use. While the acoustic coupling pad 116 may separate the sensor face 130 from the patient's body by a small distance, the various physiological signals may be effectively transmitted and received via the acoustic coupling pad 116 to the sensor portion 112 due to the acoustic pathway provided by the acoustic coupling pad 116. The features of the acoustic coupling pad 116 and the various components within the sensor portion 112 will be described in further detail later herein.

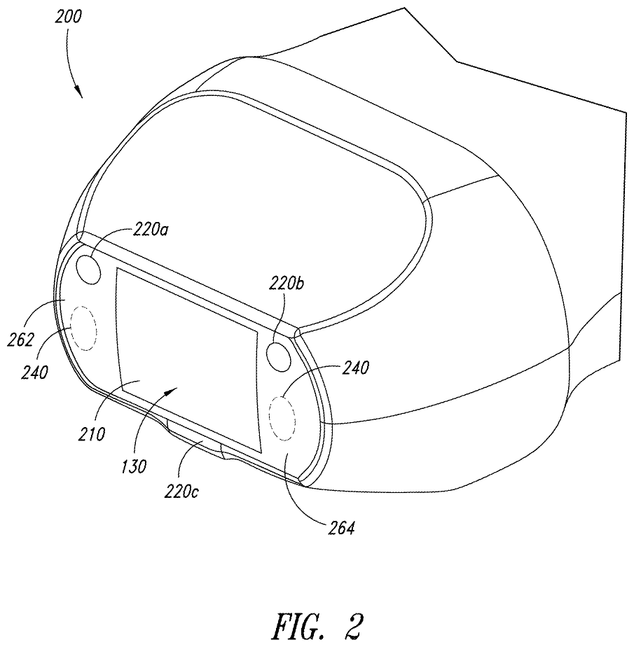

FIG. 2 is an enlarged perspective view 200 of the sensor portion 112 of the device 100 shown in FIG. 1. FIG. 2 shows the sensor portion 112 with the acoustic coupling pad 116 being detached from the sensor face 130 for illustration purposes for describing the components of the sensor portion 112 of the device 100. The sensor portion 112 of the device 100 includes an ultrasound sensor 210. In some embodiments, the sensor portion 112 includes a plurality of ECG electrodes 220a, 220b, 220c (which may be referred to collectively as an ECG sensor 220) positioned at various locations spaced apart from the ultrasound sensor 210. Any number of ECG electrodes may be included in the sensor portion 112, for example, in some embodiments the sensor portion 112 may include more than 3 ECG electrodes.

In some embodiments, the sensor portion 112 may include one or more auscultation sensors 240, e.g., a first auscultation sensor positioned near or beneath a first membrane 262 and a second auscultation sensor positioned near or beneath a second membrane 264. Each of the ultrasound sensor 210, the auscultation sensors 240, and the ECG sensor 220 is positioned adjacent to the sensor face 130 of the device 100. In use, the sensor face 130 may be placed near or in contact with a patient's skin, and the device 100 may obtain ultrasound, auscultation signals, and ECG signals via the ultrasound sensor 210, the auscultation sensors 240, and the ECG sensor 220, respectively. In some embodiments, there may be additional, various kinds of sensors incorporated in the sensor portion 112 of the device 100 to sense different physiological data according to various medical needs, and the sensors included in embodiments of the present disclosure are not limited to ultrasound sensors, auscultation sensors, and ECG sensors.

As shown in FIGS. 1 and 2, in some embodiments, the device 100 includes auscultation sensors 240 adjacent to the ultrasound sensor 210 at the sensor face 130. The auscultation sensors 240 may be any sensors operable to detect internal body sounds of a patient, including, for example, body sounds associated with the circulatory, respiratory, and gastrointestinal systems. That is, target sounds such as heart sounds of a patient may be sensed by the auscultation sensors 240. In one embodiment, the auscultation sensors 240 may be microphones. In some embodiments, the auscultation sensors 240 may be electronic or digital stethoscopes, and may include or otherwise be electrically coupled to amplification and signal processing circuitry for amplifying and processing sensed signals, as may be known in the relevant field. In another embodiment, the first auscultation sensor positioned near the first membrane 262 and the second auscultation sensor positioned near the second membrane 264 may be two identical auscultation sensors. However, in some embodiments, the device 100 may employ different kinds of auscultation sensors and the auscultation sensors may be different from one another.

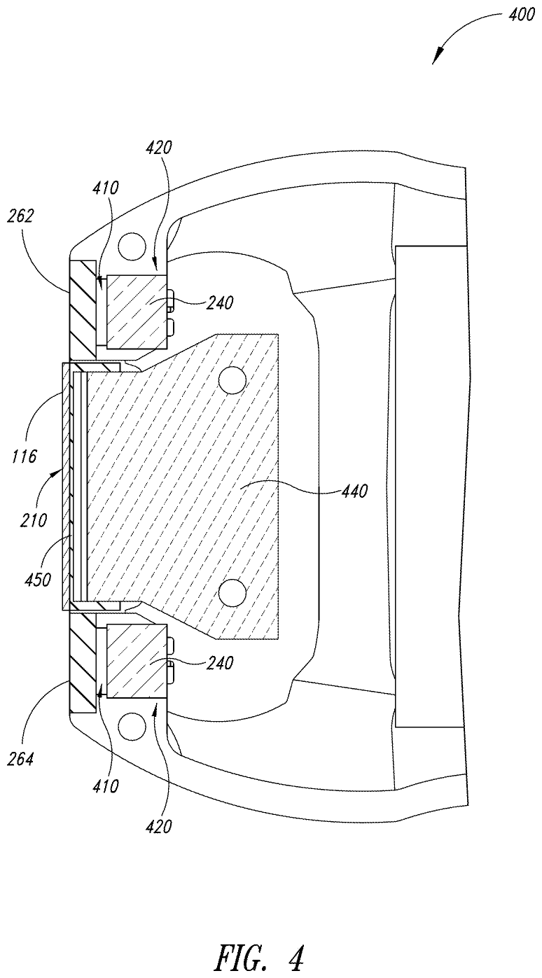

The ultrasound sensor 210 includes an ultrasound array or ultrasound transducer 440 (see FIG. 4) configured to transmit an ultrasound signal toward a target structure in a region of interest (ROI) of the patient. The transducer 440 is further configured to receive echo signals returning from the target structure in response to transmission of the ultrasound signal. To that end, the transducer 440 may include transducer elements that are capable of transmitting an ultrasound signal and receiving subsequent echo signals. In various embodiments, the transducer elements may be arranged as elements of a phased array (not shown). Suitable phased array transducers are known in the art.

The transducer 440 of the ultrasound sensors 210 may be a one-dimensional (1D) array or a two-dimensional (2D) array of transducer elements. The transducer array may include piezoelectric ceramics, such as lead zirconate titanate (PZT), or may be based on microelectromechanical systems (MEMS). For example, in various embodiments, the ultrasound sensors 210 may include piezoelectric micromachined ultrasonic transducers (PMUT), which are microelectromechanical systems (MEMS)-based piezoelectric ultrasonic transducers, or the ultrasound sensor 210 may include capacitive micromachined ultrasound transducers (CMUT) in which the energy transduction is provided due to a change in capacitance.

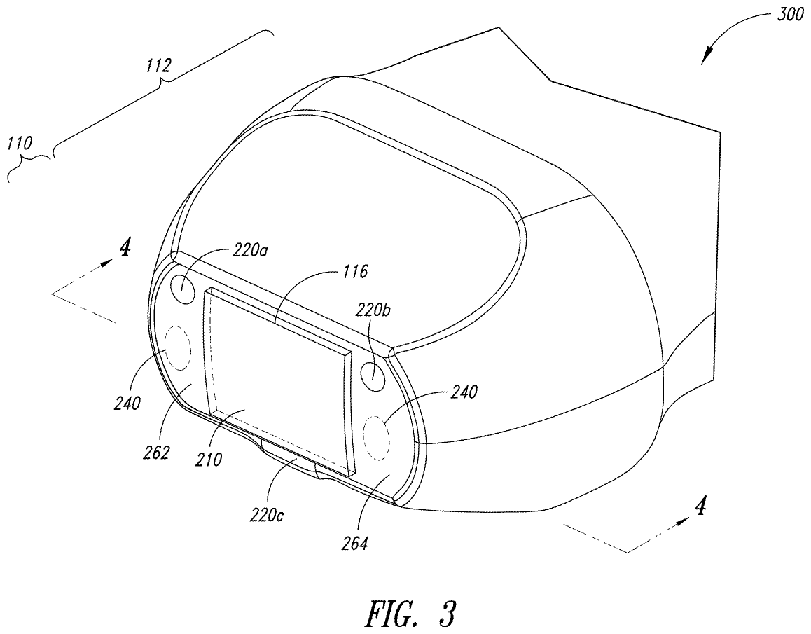

The ultrasound sensor 210 may further include an ultrasound focusing lens 450 (see FIG. 4), which is positioned distally with respect to the ultrasound transducer 440, and which may form a part of the sensor face 130. The acoustic coupling pad 116 may be disposed on the ultrasound focusing lens 450 and may replace the conventional water-based ultrasound gels which may cause the ECG electrodes 220a, 220b, 220c to be electrically connected to each other. This will be explained in more detail in relation with FIG. 3. The focusing lens 450 may be any lens operable to focus a transmitted ultrasound beam from the ultrasound transducer 440 toward a patient and/or to focus a reflected ultrasound beam from the patient to the transducer 440. The ultrasound focusing lens 450 may have a substantially flat shape as shown in FIG. 4. In some embodiments, the ultrasound focusing lens 450 may have a front surface that is substantially coplanar with the first membrane 262 and the second membrane 264. However, in other embodiments, the ultrasound focusing lens 450 may have a curved surface shape, or an oval shape. That is, the ultrasound focusing lens 450 may have different shapes depending on a desired application, e.g., a desired operating frequency, or the like. The ultrasound focusing lens 450 may be formed of any suitable material, and in some embodiments, the ultrasound focusing lens 450 is formed of a room-temperature-vulcanizing (RTV) rubber material.

The ECG sensor 220 may be any sensor that detects electrical activity, e.g., of a patient's heart, as may be known in the relevant field. For example, the ECG sensor 220 may include any number of ECG electrodes 220a, 220b, 220c, which in operation are placed in contact with a patient's skin and are used to detect electrical changes in the patient that are due to the heart muscle's pattern of depolarizing and repolarizing during each heartbeat.

As shown in FIG. 2, the ECG sensor 220 may include a first electrode 220a that is positioned adjacent to a first side of the ultrasound sensor 210 (e.g., adjacent to the left side of the ultrasound sensor 210 which may correspond to the location where the first membrane 262 is positioned), and a second electrode 220b that is positioned adjacent to a second side of the ultrasound sensor 210 that is opposite to the first side (e.g., adjacent to the right side of the ultrasound sensor 210 which may correspond to the location where the second membrane 264 is positioned). The ECG sensor 220 may further include a third electrode 220c that is positioned adjacent to a third side of the ultrasound sensor 210 (e.g., adjacent to the lower side of the ultrasound sensors 210 which is located between the first membrane 262 and the second membrane 264). This third side may extend between the first side and the second side, and a membrane adjacent to the third side may also be referred to as the third membrane (not shown). In some embodiments, the third electrode 220c may be exposed through the third membrane and the first and second electrodes 220a and 220b may be exposed through the first and second membrane 262, 264 respectively. In some embodiments, each of the first, second, and third ECG electrodes 220a, 220b, 220c have different polarities. For example, the first ECG electrode 220a may be a positive (+) electrode, the second ECG electrode 220b may be a negative (−) electrode, and the third ECG electrode 220c may be a ground electrode.

The number and positions of the ECG sensor electrodes 220 may vary in different embodiments. As shown in FIG. 2, the ECG electrodes 220a, 220b, 220c may be approximately equidistant from one another. The first and second ECG electrodes 220a, 220b may be positioned near a top edge of the sensor face 130, while the third ECG electrode 220c may be positioned between the lower side of the ultrasound sensor 210 and a bottom edge of the sensor face 130. In other embodiments, the ECG electrodes 220a, 220b, 220c the spacing between and the individual locations of the ECG electrodes 220a, 220b, 220c may be differently placed based on design needs.

In some embodiments, the ultrasound sensor 210, the ECG sensor 220, or the auscultation sensors 240 may be located differently than as shown in FIG. 2. The various sensors may be located adjacent to each other to effectively obtain the patient's physiological data but the individual sensor components can be placed in a different pattern or location. For example, depending on the specific part of the patient that is being diagnosed and according to other various medical needs, the device 100 can have auscultation sensors located only on or beneath the first membrane 262, and the ECG sensor 220 located only on or beneath the second membrane 264. In some embodiments, the ultrasound sensor 210 may be located near a first side area of the sensor face, with the auscultation sensors 240 located in the center area of the sensor face 130, and the ECG sensor 220 located near a second side of the sensor face opposite the first side. The ultrasound sensor 210, the auscultation sensors 240, and the ECG sensors 220 may be positioned in any suitable arrangement on or adjacent the sensor face 130, and embodiments provided herein are not limited to the arrangement shown in FIG. 2.

In some embodiments, first and second membranes 262, 264 are positioned adjacent to opposite sides of the ultrasound sensor 210 and may form a part of the sensor face 130. The first and second membranes 262, 264 may be formed of any suitable material, and in one embodiment, the first and second membranes 262, 264 are formed of a room-temperature-vulcanizing (RTV) rubber material. In some embodiments, the first and second membranes 262, 264 are formed of a same material as the ultrasound focusing lens 450.

In some embodiments, the sensor face 130 includes a sealant which seals the sensor face 130 of the device 100 so that it is compliant with ingress protection specifications of IPX7 of the IP Code (as published by the International Electrotechnical Commission) (e.g., it is liquid tight when submerged to a depth of at least one meter). The sealant may be provided, for example, between the membranes 262, 264 and the respective sides of the ultrasound sensor 210, and/or between the ultrasound sensor 210, the membranes 262, 264 and the side surfaces of the housing 105. In some embodiments, the sealant is provided over the ultrasound focusing lens 450 of the ultrasound sensor 210 and the membranes 262, 264. In such embodiments, the acoustic coupling pad 116 may be overlain on top of the sealant overlapping the face of the ultrasound focusing lens 450 of the ultrasound sensor 210. The sealant may be a RTV rubber material, and in some embodiments, the sealant may be formed of a same material as the ultrasound focusing lens 450 and/or the first and second membranes 262, 264.

FIG. 3 is an enlarged perspective view 300 of the pad portion 110 and the sensor portion 112 of the device 100 shown in FIG. 1, in accordance with one or more embodiments. Since most of the common elements were explained in detail in relation to FIG. 2, descriptions of previously explained elements will be omitted and the following description of FIG. 3 will focus on the features related to the pad portion 110 and the acoustic coupling pad 116.

As shown in FIG. 3, the pad portion 110 includes an acoustic coupling pad 116. The acoustic coupling pad 116 is positioned on the ultrasound focusing lens 450 of the ultrasound sensor 210. In one embodiment, the size (e.g., length and width) of the acoustic coupling pad 116 may match the size (e.g., length and width) of the ultrasound focusing lens 450 and the acoustic coupling pad 116 may be disposed on top of the lens 450. In some embodiments, the size of the acoustic coupling pad 116 may be smaller than the size of the ultrasound focusing lens 450, for example, such that the acoustic coupling pad 116 only partially covers the ultrasound focusing lens 450. In other embodiments, the size of the acoustic coupling pad 116 may be larger than the size of the ultrasound focusing lens 450, for example, such that the acoustic coupling pad 117 completely overlaps the ultrasound focusing lens 450 with portions of the acoustic coupling pad 116 extending laterally beyond side edges of the ultrasound focusing lens 450. The acoustic coupling pad 116 may have any shape or size, which may be determined based on the design needs or medical applications of the acoustic coupling pad 116 and the device 100, but will have a suitable size to provide the function of serving as an acoustic pathway for the ultrasound sensor 210. In some embodiments, the acoustic coupling pad 116 may have a suitable size to cover the ultrasound focusing lens 450, while being spaced apart from the plurality of ECG electrodes 220a, 220b, and 220c thereby preventing short circuits of the ECG electrodes 220a, 220b, and 220c through the acoustic coupling pad 116. Electrical shorts between ECG electrode leads will result in little to no ECG signals, and the size of the acoustic coupling pad 116 may be designed to not cause the short between the ECG electrode leads. This will be explained in more detail later.

The device 100 is a multifunctional device that is capable of acquiring different types of data, such as ultrasound data, auscultation data, and electrocardiography data, at the same time. The device 100 achieves this by placing various sensors (e.g., ultrasound sensor, ECG sensor, auscultation sensors) in the sensor portion 112 of the device 100. However, by placing ECG electrode leads 220a, 220b, 220c on the same surface as the ultrasound sensor 210, when the water-based ultrasound scanning gels are used for ultrasound scanning, the water-based gels may electrically connect between one or more ECG electrode leads. These unwanted connections between the ECG electrode leads 220a, 220b, 220c through the scanning gels causes the ECG signals to have noise or possibly produce unclear and incorrect ECG signals. These unclear ECG signals collected from the patient can prevent the medical practitioner from correctly diagnosing the patient based on the acquired signals. Therefore, in utilizing the device 100, the technical problem raised from using the water-based ultrasound scanning gels is overcome due to the presence of the acoustic coupling pad 116.

The proposed acoustic coupling pad 116 which serves as a replacement for the water-based gel for the ultrasound sensor 210 is placed on the ultrasound focusing lens 450 and spaced apart from the plurality of ECG electrodes 220a, 220b, 220c. In one embodiment, the plurality of ECG electrodes 220a, 220b, 220c may be disposed on the sensor face 130 and the acoustic coupling pad 116 may be placed in a location that does not electrically connect the respective ECG electrodes 220a, 220b, 220c with each other. By placing the acoustic coupling pad 116 over the ultrasound focusing lens 450 while spacing the acoustic coupling pad 116 away from the plurality of ECG electrodes 220a, 220b, 220c, the positional relationship ensures that the ECG electrodes will not be electrically connected to each other. Also at the same time, the acoustic coupling pad 116 may provide the ultrasound sensor 210 with an acoustic pathway for improving the reception of ultrasound data of the patient. The acoustic coupling pad 116 eliminates the air gap that may be formed between the ultrasound sensors 210 and the patient's skin and transfers ultrasound signals with minimum or reduced acoustic loss.

In some embodiments, the acoustic coupling pad 116 may have properties for providing adequate ultrasound coupling. These properties ensure that the ultrasound signals from the patient will be properly obtained from the acoustic coupling pad 116 to the ultrasound sensor 210 with high quality ultrasound image. In one embodiment, the acoustic coupling pad 116 may be an acoustically transparent silicone gel pad. For example, the acoustically transparent silicone gel pad has shown promising results of increasing ultrasound sensitivity as compared to the ultrasound gels and eliminated the need to use ultrasound gels. In some embodiments, synthetic rubber may be used in forming the acoustic coupling pad 116. The synthetic rubber may include substances such as cis-1,4-polybutadiene for the acoustic coupling pad 116, which has been shown to reduce acoustic loss. The acoustic coupling pad 116 formed utilizing these materials has the capability of clearly transmitting the ultrasound signals from the patient's bodily organs to the ultrasound sensor 210 of the device 100 with minimum or low acoustic loss and the device 100 is able to clearly amplify and cancel any noise from the signals to reproduce a definite ultrasound image.

In some embodiments, the acoustic coupling pad 116 may be formed using materials taking into account the appropriate acoustic impedance for the specific ROI of the patient to be imaged (e.g., certain tissues such as heart, kidney, liver, muscle, etc.). The acoustic impedance may be based on the density of a certain tissue and the speed of sound within that tissue. The acoustic impedance of a tissue or material such as blood, fat, liver, heart, brain, kidney, muscle, etc., may all differ. A typical density, speed of sound, and acoustic impedance values of various tissues or materials are shown in Table 1.

TABLE 1
Examples of Typical Density, Speed of Sound, and
Acoustic Impedance Values of Tissues/Materials

| | | Speed of | Acoustic |
|---|---|---|---|
| Tissue or | Density | Sound | Impedance |
| Material | (g/cm³) | (m/sec) | [kg/(sec · m²)] × 10⁶ |
| Water | 1 | 1480 | 1.48 |
| Brain | 1.03 | 1550 | 1.60 |
| Heart | 1.045 | 1570 | 1.64 |
| Kidney | 1.05 | 1570 | 1.65 |
| Liver | 1.06 | 1590 | 1.69 |

Accordingly, based on which ROI of the patient being examined, the acoustic coupling pad 116 may be variously designed so that the acoustic impedance of the acoustic coupling pad 116 is matched or is substantially similar to the acoustic impedance of tissue between the acoustic coupling pad 116 and a particular structure or organ to be imaged.

In general, a portion of ultrasound energy output by an ultrasound imaging device is reflected at any interface between media having different acoustic impedances. The difference in acoustic impedance between the patient's skin and the outer surface of an ultrasound imaging device which contacts the patient's skin therefore at least partially dictates how much ultrasound energy will be transmitted into and out of the patient, as well as how much of the ultrasound energy will be reflected at the interface with the patient's skin. In some embodiments, the acoustic coupling pad 116 may be formed to have an impedance that is substantially the same or similar to an impedance of human tissue, which facilitates efficient transmission of the ultrasound energy through the tissue (which may include, for example, skin, fat, water, etc.) and to a desired structure of the patient to be imaged. For example, by adjusting the ratio or amount of cis-1,4-polybutadiene in the synthetic rubber which may be utilized in the acoustic coupling pad 116, the acoustic impedance of the acoustic coupling pad 116 may be formed to substantially match the impedance of the patient's skin, thereby reducing or minimizing undesired reflection of ultrasound energy at the interface between the acoustic coupling pad 116 and the patient's skin. This may ensure efficient transmission of the ultrasound energy through the skin and tissue, and reduce or minimize loss (e.g., reflection) of the acoustic signals as they are transmitted through the skin and tissue toward and from a particular structure or organ under diagnosis.

In FIGS. 3, 4 and 5, the acoustic coupling pad 116 has been described as being a thin rectangular pad, or a rectangular pad that has a round corner on the edges to have a cylindrical edge. However, the shape of the acoustic coupling pad 116 is not limited to these shapes and the acoustic coupling pad 116 may have various shapes according to design needs. For example, the acoustic coupling pad 116 may be of a circular pad shape, triangular shape, or polygonal shape, etc. In other embodiments, the shape of the acoustic coupling pad 116 may depend on the shape of the lens 450.

In some embodiments, the acoustic coupling pad 116 may be a silicone pad or a synthetic rubber pad including cis-1,4-polybutadiene with a thickness less than 10 mm. More preferably, the acoustic coupling pad 116 may be made of a silicone pad or a synthetic rubber pad including cis-1,4-polybutadiene and may have a thickness less than 6 mm. In one embodiment, the height of the acoustic coupling pad 116 may be measured from the distance between a first surface (e.g., top surface) and a second surface (e.g., bottom surface) of the acoustic coupling pad 116. In another embodiment, the height of the acoustic coupling pad 116 may be measured from the surface of the lens 450 in which the acoustic coupling pad 116 is disposed over to the first surface (e.g., top surface) of the acoustic coupling pad 116. Since the human skin that will contact the acoustic coupling pad 116 is generally soft, elastic and curvy, the acoustic coupling pad 116 may be formed to have an oval shape. For example, the acoustic coupling pad 116 may be of a convex shape where the center of the top surface is protruding outwards. In this example, the height of the acoustic coupling pad 116 may be determined based on the distance between the central point of the top convex surface to the top surface of the lens 450. On the other hand, the acoustic coupling pad 116 may be of a concave shape where the center of the top surface is protruding inwards (towards more closer to the lens 450). In this example, the height of the acoustic coupling pad 116 may be determined based on the distance between the central point of the top concave surface to the top surface of the lens 450. In this particular example, due to the concave shape of the acoustic coupling pad 116, the height in the periphery of the pad 116 will be higher than the height in the center of the pad 116. However, in some embodiments, the height of the pad 116 may be determined based on the central point of the concave shaped pad.

The thickness of the acoustic coupling pad 116 needs to take into account that if the pad is too thick, it may space the ECG sensor 220 apart from the patient's skin, thereby limiting the detection of adequate ECG signals. As such, the thickness of the acoustic coupling pad 116 may be designed to ensure that the device 100, when in use would allow the plurality of ECG electrodes 220a, 220b, 220c on the sensor face 130 to touch the skin of the patient. Since skin is soft and elastic, even though the ECG electrodes 220a, 220b, 220c may be spaced apart from the exposed surface of the acoustic coupling pad 116, when the sensor face 130 is applied to the patient's skin with a small amount of force, the ECG electrode leads 220a, 220b, 220c may still contact the patient's skin, ensuring accurate measure of ECG signals. For example, with the acoustic coupling pad 116 having a thickness of 10 mm or less, the ECG electrode leads 220a, 220b, and 220c can contact the patient's skin and can appropriately and accurately obtain ECG signals of the patient, while the acoustic coupling pad 116 also contacts the skin such that the ultrasound sensor 210 can acquire ultrasound signals/images through the acoustic coupling pad 116.

An adhesive may be applied between the acoustic coupling pad 116 and the ultrasound focusing lens 450 to improve the mechanical coupling of the acoustic coupling pad 116 to the lens 450. For example, a light adhesive may be used to couple the acoustic coupling pad 116 with the ultrasound focusing lens 450. When the adhesive is applied at one side of the acoustic coupling pad 116 (e.g., the array or transducer side 440), the acoustic coupling pad 116 may cover the ultrasound focusing lens 450 or even the auscultation sensors 240. However, the adhesive does not cover the ECG electrode leads 220a, 220b, 220c so that the acoustic coupling pad 116 is overlain over the ECG electrode leads 220a, 220b, 220c which may cause unwanted electrical short circuits.

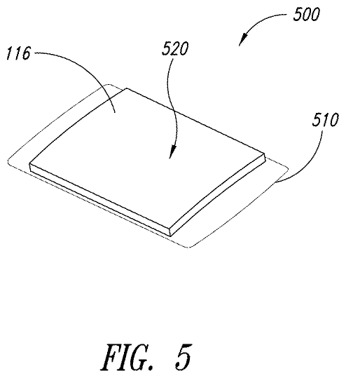

The exposed side of the acoustic coupling pad 116 (e.g., the side that directly contacts the patient) may be coated with a biocompatible coating material, which may improve lubricity and coupling with the ROI of the patient. This will be explained in more detail in relation with FIG. 5.

FIG. 4 is a cross-sectional view 400 taken along the cut-line 4-4 of FIG. 3, illustrating further details of the pad portion 110 and the sensing portion 112 of the device 100, in accordance with one or more embodiments.

As shown in FIG. 4, the first and second membranes 262, 264 are positioned in front of the auscultation sensors 240 and adjacent to the ultrasound focusing lens 450. The acoustic coupling pad 116 is on the ultrasound focusing lens 450 of the ultrasound sensors 210. In some embodiments, the auscultation sensors 240 are spaced apart from the membranes 262, 264 by respective gaps 410, which may be air gaps. These air gaps may provide an acoustic tunnel for clearly receiving the auscultation data through the auscultation sensors 240.

The auscultation sensors 240 may be positioned in respective auscultation sensor sockets 420, which may fix a position of the auscultation sensors 240 so that they are spaced apart from the respective membranes 262, 264 by a desired gap 410. In some embodiments, the gaps 410 have a distance within a range of about 0.5 mm to about 1.5 mm. In some embodiments, the gaps 410 have a distance of about 1 mm. In some embodiments, the auscultation sensor sockets 420 are formed as an internal piece of the housing 105. For example, the auscultation sensor sockets 420 may be molded into the housing 105. The auscultation sensor sockets 420 may be sized to accommodate the auscultation sensors 240, and the auscultation sensors 240 may be securely held in the auscultation sensor sockets 420. In some embodiments, the auscultation sensors 240 may be secured within the auscultation sensor sockets 420 by an adhesive material.

The auscultation sensor sockets 420 may fasten or affix the auscultation sensors 240 to the housing 105 so that it impedes any movement of the auscultation sensors 240 in any direction. If there is a room or gap between the auscultation sensor sockets 420 and the auscultation sensors 240, this room or gap may create unnecessary noises that are irrelevant to the physiological signals or sounds of the patient. The fixed position of the auscultation sensors 240 eliminates any movements so that the auscultation sensors 240 can clearly obtain the physiological signals or sounds of the patient during use.

In addition, in some embodiments, with the auscultation sensors 240 positioned in the auscultation sensor sockets 420 and spaced apart from the membranes 262, 264 by a desired gap 410, the membranes 262, 264 may operate as diaphragms which convert mechanical vibrations (e.g., from motion against the membranes 262, 264 and/or in response to receiving acoustic vibrations) into sounds which are detectable by the auscultation sensors 240.

In one embodiment, the first and second membranes 262, 264 are positioned adjacent to opposite sides of the ultrasound sensor 210 and may form a part of the sensor face 130. The first and second membranes 262, 264 may be formed of any suitable material, and in one embodiment, the first and second membranes 262, 264 are formed of a room-temperature-vulcanizing (RTV) rubber material. In some embodiments, the first and second membranes 262, 264 are formed of a same material as the ultrasound focusing lens 450.

In some embodiments, the ultrasound focusing lens 450 may be substantially coplanar with the first membrane 262 and the second membrane 264. By positioning the ultrasound focusing lens 450 in the same plane as the first and second membrane 262, 264, the distance between the ECG electrode leads 220a, 220b, 220c also positioned on the first and second membrane 262, 264 and the patient's skin can be maintained at a desired, suitable distance even after the acoustic coupling pad 116 is attached to the ultrasound focusing lens 450. If the distance between the outer surface of the acoustic coupling pad 116 that is in contact with the patient's skin and the plane of lens 450 (which may be coplanar with the first and second membrane 262, 264) is spaced apart beyond a suitable distance, the ECG electrode leads 220a, 220b, 220c may not directly contact the patient's skin which may prevent the leads from effectively receiving ECG data.

In other embodiments, the ultrasound focusing lens 450 may be placed to so that the acoustic coupling pad 116 may be substantially coplanar with the first membrane 262 and the second membrane 264. By placing the ultrasound focusing lens 450 so that the acoustic coupling pad 116 is in the same plane as the first and second membranes 262, 264, the ECG electrode leads 220a, 220b, 220c and the acoustic coupling pad 116 may directly contact the patient's skin without applying any additional force to reduce a gap between the ECG electrode leads 220a, 220b, 220c and the patient's skin. This configuration may increase the quality of the ECG data received from the ECG electrode leads 220a, 220b, 220c since there will be no air gap between the leads 220a, 220b, 220c and the patient's skin. The ultrasound focusing lens 450 may be recessed in a direction towards the ultrasound transducer 440 which may decrease the space between the lens 450 and the transducer 440. For example, the ultrasound focusing lens 450 may be recessed with respect to the membranes 262, 264 by a distance that is about the same as the thickness of the acoustic coupling pad 116. In one embodiment, the acoustic coupling pad may have a thickness of about 5 mm. In this embodiment, the lens 450 may be recessed with respect to outer or exposed surfaces of the membranes 262, 264 by a distance of about 5 mm. When the acoustic coupling pad 116 is attached to the lens 450, the outer surface of the acoustic coupling pad 116 may be substantially coplanar with the outer surfaces of the first and second membranes 262, 264 and the ECG electrode leads 220a, 220b, 220c may directly contact the patient's skin to provide improved acquisition of ECG data. While providing an entirely coplanar surface at the sensor face 130 may be beneficial in the reception of the patient's physiological data, due to the soft and cushion-like surface of the human skin, in some embodiments, the impact of the spaced distance between the plane of the membranes 262, 264 and the acoustic coupling pad 116 may have minimum impact on the quality of the ECG data received through the ECG electrode leads 220a, 220b, 220c.

FIG. 5 is a perspective view 500 of an acoustic coupling pad 116, in accordance with one or more embodiments.

As shown in FIG. 5, an acoustic coupling pad 116 is placed on a backing 510. The backing 510 is adhered to a first surface (e.g., a surface that directly faces and contacts the backing 510) of the acoustic coupling pad 116 with an adhesive material. The adhesive material may remain on the acoustic coupling pad 116 as an adhesive layer after the acoustic coupling pad 116 is peeled off from the backing 510. The adhesive material forms a film-like thin adhesive layer on the first surface of the acoustic coupling pad 116 and this material may be any suitable material that can enhance the mechanical or physical coupling between the ultrasound focusing lens 450 and the acoustic coupling pad 116. That is, the backing 510 may leave adhesives on the first surface of the acoustic coupling pad 116 that can be easily attached with the ultrasound focusing lens 450 which is formed of a room-temperature-vulcanizing (RTV) rubber material. In addition, these adhesive materials may be any suitable materials having characteristics that are strong enough to be coupled with the lens 450 but is capable of being easily peeled off by a medical practitioner after use or after the diagnosis is completed. Some examples of adhesive materials which may be provided on the first surface of the acoustic coupling pad 116 may include, but is not limited to, tape, paste, glue, or any other suitable material.

The acoustic coupling pad 116 includes a second surface 520, that is opposite of the first surface. In some embodiments, the second surface 520 may be parallel to the first surface. However, in other embodiments, depending on the shape of the acoustic coupling pad 116, the second surface 520 is not necessarily parallel to the first surface and the second surface 520 may have a curvature depending on the various application and design needs of the acoustic coupling pads. For example, while the first surface may have a flat surface to improve the adhesion with the lens 450, the second surface 520 may have a wave-shape surface to improve smoothness or lubricity with the patient's skin during ultrasound imaging.

The second surface 520 directly contacts the patient or the patient's skin during use of the device 100. When the device 100 is in use, the second surface 520 contacts the skin or surface of the region that is to be diagnosed or imaged. The second surface 520 of the acoustic coupling pad 116 may be coated with a biocompatible coating, which may be a coating of any biocompatible material which is compatible with living tissue and which does not produce a toxic or immunological response when exposed to the body. Moreover, the biocompatible coating may decrease friction between the acoustic coupling pad 116 and the patient's skin. The biocompatible coating may be provided as a thin film-like layer on the second surface 520 of the acoustic coupling pad 116. In one embodiment, the biocompatible coating is provided to improve lubricity of the acoustic coupling pad 116. Biocompatible coatings may include substances having smooth and slippery oil-like materials. These biocompatible coatings normally do not have any impact or effect that will alter or change the physiological data (e.g., ultrasound data). That is, the ultrasound data received through the ultrasound sensor 210 may not be affected by the biocompatible coating applied on the second surface 520 of the acoustic coupling pad 116. The biocompatible coating may be a medical grade coating that serves as an acoustic channel that will easily pass through any ultrasound signals to and from the ultrasound transducers 440. The biocompatible coating is capable of relaying the ultrasound signals with minimum acoustic loss or no acoustic loss. In other embodiments, the biocompatible coating may have hydrophilic characteristics. In another embodiment, the biocompatible coating may have abrasion resistant characteristics. For example, the biocompatible coating may include any bio-coating material that is IEC10993 compliant. Further examples may include, but are not limited to, silicone-based biocompatible materials, biocompatible polymers, synthetic polymers, phenolic resin and the like.

In some embodiments, as long as the acoustic coupling pad 116 has a shape to provide the lens 450 of the ultrasound sensors 210 with a non acoustic-loss pathway, the acoustic coupling pad 116 may be of a circular pad shape, triangular shape, or polygonal shape, etc. In other embodiments, the shape of the acoustic coupling pad 116 may depend on the shape of the lens 450 and the area that the acoustic coupling pad 116 needs to cover. In some embodiments, the bottom surface of the acoustic coupling pad 116 (e.g., the first surface) may be a flat surface and the top surface of the acoustic coupling pad 116 (e.g., the second surface 520) may be a wave-shaped or wavy surface. The acoustic coupling pad 116 may have various shapes and sizes depending on the application and the design needed.

In one embodiment, the acoustic coupling pad 116 is a silicone pad. For example, this silicone pad may be a silicone that is IEC10993 compliant. However, the acoustic coupling pad 116 is not limited to these silicone pads. In other embodiments, the acoustic coupling pad 116 may be a synthetic rubber pad including cis-1,4-polybutadiene. In some embodiments, the acoustic coupling pad 116 may be formed with any material that has characteristics of minimum or low acoustic loss which is capable of relaying the ultrasound signals to produce a quality ultrasound image.

The thickness of the acoustic coupling pad 116 can be manufactured to have a thickness less than 10 mm. More preferably, the acoustic coupling pad 116 may have a thickness less than 6 mm. In one embodiment, the thickness of the acoustic coupling pad 116 may be measured from a distance between the first surface (e.g., the surface adhered to the backing 510) and the second surface 520 of the acoustic coupling pad 116. In use, the acoustic coupling pad 116 may contact human skin, which is generally soft, elastic and curvy. Accordingly, the acoustic coupling pad 116 may be formed to have an oval shape. For example, the acoustic coupling pad 116 may be of a convex shape where the center of the second surface 520 is protruding outwards (direction opposite of the backing 510). In this example, the height of the acoustic coupling pad 116 may be determined based on the distance between the central point (or the highest point) of the top convex surface (e.g., second surface 520 with a convex surface) to the surface of backing 510. On the other hand, the acoustic coupling pad 116 may be of a concave shape where the center of the second surface 520 is protruding inwards (direction towards the backing 510). In this example, the height or the thickness of the acoustic coupling pad 116 may be determined based on the distance between the central point (or the lowest point) of the top concave surface (e.g., second surface 520 with a concave surface) to the surface of the backing 510. In this particular example, due to the concave shape of the acoustic coupling pad 116, the thickness in the periphery of the pad 116 will be thicker than that in the center of the pad 116. Based on various needs, the thickness of the pad 116 may be determined based on multiple points of the concave shaped pad or other shaped pads.

In some embodiments, the acoustic coupling pad 116 may be labeled with a radio-frequency identification tag (RFID) to ensure that the acoustic coupling pad 116 is not used multiple times. The RFID tag attached to the acoustic coupling pad 116 may use electromagnetic fields to easily and automatically identify and track the use of the acoustic coupling pad 116. The RFID tags attached contain electronically stored information. Examples of electronically stored information may include information indicating when the acoustic coupling pad 116 was first manufactured, whether the acoustic coupling pad 116 has been used before or not, the location (e.g., hospital or other medical organization) where the pad 116 was used, and which medical practitioner used the acoustic coupling pad 116 to diagnose a patient, etc. The RFID tag may be provided on any surface of the acoustic coupling pad 116, or may be embedded within the acoustic coupling pad 116. The RFID tag can be provided in any suitable location in the acoustic coupling pad 116 which does not affect or otherwise impede the transmission of the ultrasound signals. For example, the RFID tag may be located in a side surface of the acoustic coupling pad 116 or in the bottom surface of the acoustic coupling pad 116 to not hinder the transfer of ultrasound signals to and from the transducers 440. In other embodiments, a barcode may be used in place of RFID tags. In further embodiments, any form of codes, identification tags capable of being read by a machine-readable and utilizes encoded or encrypted symbols may be used and the sources for identifying is not necessarily limited to barcodes and RFID tags.

The various embodiments described above can be combined to provide further embodiments. All of the U.S. patent applications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A device, comprising:
an ultrasound sensor on a sensor face of the device;
an electrocardiogram (ECG) sensor on the sensor face of the device; and
an acoustic coupling pad on the ultrasound sensor, the ECG sensor being spaced apart from the acoustic coupling pad.

2. The device of claim 1 wherein the ultrasound sensor includes an ultrasound transducer array and an ultrasound lens on the ultrasound transducer array, wherein the acoustic coupling pad is removably attached to the ultrasound lens.

3. The device of claim 2 wherein the ECG sensor includes:
a first ECG electrode adjacent to a first side of the ultrasound sensor;
a second ECG electrode adjacent to a second side of the ultrasound sensor that is opposite the first side; and
a third ECG electrode adjacent to a third side of the ultrasound sensor, the third side extending between the first side and the second side,
wherein the acoustic coupling pad is spaced apart from and electrically isolated with respect to each of the first, second and third ECG electrodes.

4. The device of claim 3, further comprising:
a first membrane adjacent to the first side of the ultrasound sensor, the first ECG electrode being exposed through the first membrane;
a second membrane adjacent to the second side of the ultrasound sensor, the second ECG electrode being exposed through the second membrane; and
a third membrane adjacent to the third side of the ultrasound sensor, the third ECG electrode being exposed through the third membrane,
wherein the first membrane, the second membrane, and the third membrane form respective portions of the sensor face.

5. The device of claim 4, wherein the first, second, and third membranes and a surface of the ultrasound lens are coplanar to each other, and a height of the acoustic coupling pad from the surface of the ultrasound lens is less than about 10 mm.

6. The device of claim 4 wherein the ultrasound lens, the first membrane, and the second membrane include a room-temperature-vulcanizing rubber material.

7. The device of claim 3 wherein the acoustic coupling pad includes:
a biocompatible coating layer on a first surface of the acoustic coupling pad; and
an adhesive layer on a second surface of the acoustic coupling pad opposite the first surface, the adhesive layer in contact with at least a portion of the ultrasound lens,
wherein the adhesive layer is spaced apart from the ECG sensors.

8. The device of claim 7 wherein a thickness of the acoustic coupling pad between the first surface and the second surface is equal to or less than 6 mm.

9. The device of claim 1 wherein the acoustic coupling pad includes at least one of silicone or synthetic rubber.

10. The device of claim 9 wherein the synthetic rubber includes cis-1,4-polybutadiene.

11. An acoustic coupling pad for an ultrasound device, comprising:
an acoustically conductive body having a first surface and a second surface opposite the first surface;
a biocompatible coating layer on the first surface; and
an adhesive layer on the second surface.

12. The acoustic coupling pad of claim 11, wherein the biocompatible coating layer includes biocompatible silicone.

13. The acoustic coupling pad of claim 11, wherein a thickness of the acoustic coupling pad between the first surface and the second surface is less than 10 mm.

14. The acoustic coupling pad of claim 13, wherein the thickness of the acoustic coupling pad between the first surface and the second surface is less than 6 mm.

15. The acoustic coupling pad of claim 11, wherein the acoustically conductive body includes a synthetic rubber.

16. The acoustic coupling pad of claim 15, wherein the synthetic rubber includes cis-1,4-polybutadiene.

17. The acoustic coupling pad of claim 11, further comprising a backing, the acoustic coupling pad being removably secured to the backing by the adhesive layer.

18. An ultrasound probe, comprising:
a housing;
a sensor face exposed at one end of the housing;
an ultrasound transducer array;
an ultrasound lens on the ultrasound transducer array and adjacent to the sensor face; and
an acoustic coupling pad removably attached to the ultrasound lens.

19. The ultrasound probe of claim 18 wherein the ultrasound lens defines at least a portion of the sensor face of the ultrasound probe, and the acoustic coupling pad extends outwardly beyond the sensing face.

20. The ultrasound probe of claim 18 wherein the ultrasound lens is recessed with respect to the sensor face of the ultrasound probe.

21. The ultrasound probe of claim 18, further comprising an electrocardiogram (ECG) sensor on the sensor face.
